(19) United States
(12) Patent Application Publication
(10) Pub. No.: US 2012/0221042 A1
(43) Pub. Date: Aug. 30, 2012

(54) Methods And Devices For Treating The Left Atrial Appendage

(75) Inventors: Robert S. Schwartz, Inver Grove Heights, MN (US); Robert A. Van Tassel, Excelsior, MN (US); Gregg S. Sutton, Maple Grove, MN (US); Eric J. Simso, Minnetrista, MN (US)

(73) Assignee: TriCardia, LLC

(21) Appl. No.: 13/361,871

(22) Filed: Jan. 30, 2012

Related U.S. Application Data
(60) Provisional application No. 61/437,488, filed on Jan. 28, 2011.
(60) Provisional application No. 61/480,201, filed on Apr. 28, 2011.

(51) Int. Cl. A61B 17/08

(52) U.S. Cl. 606/213

(57) ABSTRACT

Methods and devices that treat the left atrial appendage by bringing the distal wall of the appendage to a position where the tissue of the wall blocks the ostium, thereby preventing blood from flowing into the appendage. The methods and devices are adapted to create a separation between the distal wall of the appendage and the adjacent pericardium such that the risk of rupturing the pericardium are minimized.

### RELATED APPLICATIONS

This application claims priority to U.S. Provisional Application Ser. No. 61/437,488 filed Jan. 28, 2011 entitled Methods And Devices For Treating The Left Atrial Appendage, and to U.S. Provisional Application Ser. No. 61/480,201 filed Apr. 28, 2011 entitled LAA Closure Device And Method, both of which are hereby incorporated herein by reference in their entireties.

### BACKGROUND OF THE INVENTION

The left atrial appendage, or “LAA” as it will be referred to herein, is a structure that gives rise to stagnant blood flow and thrombus formation, especially in patients suffering from atrial fibrillation. It has been approximated that 90% of blood clots developed in atrial fibrillation cases are formed in the LAA. As the function of the LAA is thought to be mainly embryonic, one solution is to surgically remove the atrial appendage. However, surgery is always associated with risks due to infection, bleeding, incomplete ablation, etc.

Efforts have been directed lately toward various devices and techniques to percutaneously ablate, close or occlude the LAA. Examples of such devices include the WATCHMAN device developed by Atritech Inc., of Plymouth, Minn. and the PLAATO device developed by Appriva Medical, Inc., of Sunnyvale, Calif. Many of the occlusive devices, however, have been relatively stiff, causing problems with pericardial effusion, embolization, incomplete LAA closure, device dislodgement, and a loss of device integrity. Moreover, all such devices leave a large, prosthetic surface at the ostium of the LAA, which can be thrombogenic, cause irritation, or inflammation.

There is thus a need for a method and device useable to prevent blood from stagnating in the LAA, while having a compliance that does not interfere with normal blood flow through the left atrium, and does not result in pericardial effusion, embolization, and a loss of device integrity. There is also a need for a device that permits ostial ablation and presents an immediate and longterm tissue interface to the left atrial blood.

### SUMMARY OF THE INVENTION

Several embodiments are shown and described herein directed to percutaneously or surgically preventing the stagnation of blood in the LAA. The methods and devices are generally directed to accomplishing the steps of accessing the LAA, either surgically by endocardial or epicardial means or navigating percutaneously to the LAA; entering the LAA via the ostium of the LAA or externally accessing the LAA in an epicaridal surgical approach; creating separation between the outside wall of the LAA and the pericardium; deploying a distal retraction device between the LAA and the pericardium; and reducing or eliminating the LAA volume by pulling the wall of the LAA toward the LAA ostium and securing the LAA wall in place to block the ostium, leaving a tissue surface to ablate the LAA ostium both immediately and in the long term.

For example, one embodiment of the method of the invention involves treating a left atrial appendage of a patient by navigating a catheter to the LAA via the left atrium of a patient; creating a space between the LAA and the pericardium of the patient adjacent the LAA; deploying an expandable device inside the space created; reducing an internal volume of the LAA by retracting the tissue of the LAA toward the ostium of the LAA; securing the expandable device such that said internal volume of the LAA remains reduced; and releasing the expandable device.

Another embodiment of the method of the invention involves treating a left atrial appendage of a patient during an open heart procedure by accessing the LAA via the left atrium, deploying an anchor device on the epicardial surface of the LAA, and reducing an internal volume of the LAA by retracting the tissue of the LAA toward the ostium of the LAA; securing the expandable device such that said internal volume of the LAA remains reduced; and releasing the expandable device.

A third embodiment of the method of the invention involves treating a left atrial appendage of a patient during a closed-heart procedure such as CABG. In this embodiment, the ostium of the LAA would be accessed and an expandable anchor device depoloyed at the ostium and again retracting the LAA tissue against the ostium of the LAA, such that said internal volume of the LAA remains reduced; and releasing the expandable device

Using the posterior LAA wall to block the ostium overcomes many of the problems with prior art attempts to occlude or close the LAA as the LAA wall is native tissue, which flexes with the contractions of the heart and presents no risk of fatigue over time. This tissue interface is immediately in place, obviating the need for anticoagulation measures after implant.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a cutaway view of the left side of a heart with a component of the invention entering the left atrium through the foramen ovale;

FIG. 2 is a perspective view of an embodiment of a component of the invention entering an LAA;

FIG. 3 is a perspective view of an embodiment of a component of the invention entering an LAA;

FIG. 4 is a partial perspective view of an embodiment of a component of the invention;

FIG. 5 is a partial perspective view of an embodiment of a component of the invention;

FIG. 6 is a partial perspective view of an embodiment of a component of the invention;

FIG. 7 is a partial perspective view of an embodiment of a component of the invention;

FIG. 8 is a partial side view of an embodiment of a component of the invention;

FIG. 9 is a partial side view of an embodiment of a component of the invention;

FIG. 10 is a partial side view of an embodiment of a component of the invention;

FIG. 11 is a partial perspective view of an embodiment of a component of the invention;

FIG. 12 is a partial side view of an embodiment of a component of the invention;

FIG. 13 is a partial perspective view of an embodiment of a component of the invention;

FIG. 14 is a partial perspective view of an embodiment of a component of the invention;

FIG. 15 is a partial perspective view of an embodiment of a component of the invention;

FIG. 16 is a partial perspective view of an embodiment of a component of the invention;

FIG. 17 is a partial perspective view of an embodiment of a component of the invention;

FIG. 18 is a partial perspective view of an embodiment of a component of the invention;

FIG. 19 is an axial view of an embodiment of a component of the invention;

FIG. 20 is a axial view of an embodiment of a component of the invention;

FIG. 21 is a partial side view of an embodiment of a component of the invention;

FIG. 22 is a partial side view of an embodiment of a component of the invention;

FIG. 23 is a partial perspective view of an embodiment of a component of the invention;

FIG. 24 is a partial perspective view of an embodiment of a component of the invention;

FIG. 25 is a partial perspective view of an embodiment of a component of the invention;

FIG. 26 is a partial perspective view of an embodiment of a component of the invention;

FIG. 27 is a partial perspective view of an embodiment of a component of the invention;

FIG. 28 is a partial perspective view of an embodiment of a component of the invention; and,

FIG. 29 is a perspective view of a deployed configuration of the component of FIG. 28.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

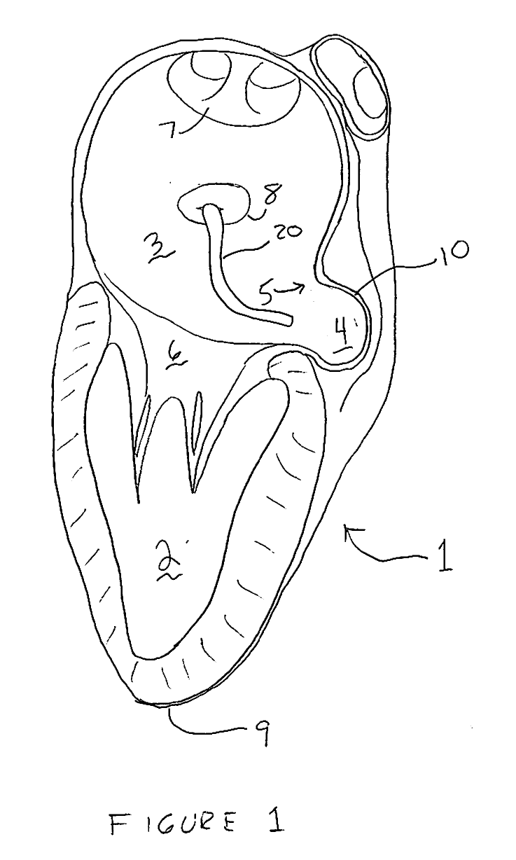

Referring now to the figures, and first to FIG. 1, there is shown a cut away view of the left side of the human heart 1, including the left ventricle 2, the left atrium 3, the LAA 4, the ostium 5 of the LAA, the mitral valve 6, and the right pulmonary veins 7. Below the right pulmonary veins 7, there is shown the foramen ovale 8, which in normally developed hearts is closed. It is merely presented to show a potential entry point to the left atrium 3. The oval labeled as the foramen ovale 8 is not to be interpreted as an open defective septum between the left and right atria.

Accessing the LAA

Via Catheter Navigation

The first step in the method of the present invention is to navigate to the left atrium 5. In one embodiment, this is accomplished with a delivery catheter 20 of the present invention, discussed in more detail below. Navigation to the left atrium 3 is accomplished using any of a variety of known or unknown methods, including but not limited to, the use of guide wires and/or steerable catheters, or surgery, and using as an entry point the foramen ovale 8, or the apex 9 of the heart.

An embodiment of a catheter 20 is shown in FIG. 1, entering the left atrium 3 through the foramen ovale 8. Doing so creates a small puncture wound through the foramen ovale 8, which will typically heal in a short time without complication. The catheter 20 is then navigated to the ostium 5 of the LAA 4.

Via Endocardial Surgery

The first step in the alternative endocardial surgical approach is to access the ostium of the LAA via the left atrium. This could be performed concomitant to a separate surgical procedure such as mitral valve repair or surgical ablation. Since the pericardium is already retracted, puncturing the opposite surface of the LAA is straightforward and does not require a separate step to create pericardial separation (below). The rest of the procedure would be similar to the percutaneous approach described below but without the need to puncture the pericardium or separately engage the LAA tissue.

Via Epicardial Surgery

The first step in the alternative epicardial surgical approach is to access the LAA directly, and puncture the LAA to gain access to the ostium of the LAA. This could be performed concomitant to a separate surgical procedure such as Coronary Artery Bypass Surgery (CABG). Since the pericardium is already retracted, puncturing the surface of the LAA is straightforward and does not require a separate step to create pericardial separation. In this iteration an anchor is deployed first over the ostium of the LAA. What is the proximal anchor in the percutaneous or endocardial approach becomes the distal anchor and the distal retraction device becomes in effect a proximal retraction device. The rest of the procedure would be similar to the percutaneous or endocardial approach described below.

Creating Pericardial Separation

The next step of an embodiment of the method of the invention is creating a separation between the wall 10 of the LAA 4 and the pericardium 9 (shown in FIG. 2). This separation is desired prior to deployment of a device in order to mitigate the risk of puncturing the pericardium, which may result in damage to the pericardium. A worse result of puncturing the pericardium may be accidental deployment of the device outside of the pericardium, resulting in a possible pericardial tear during the retraction step.

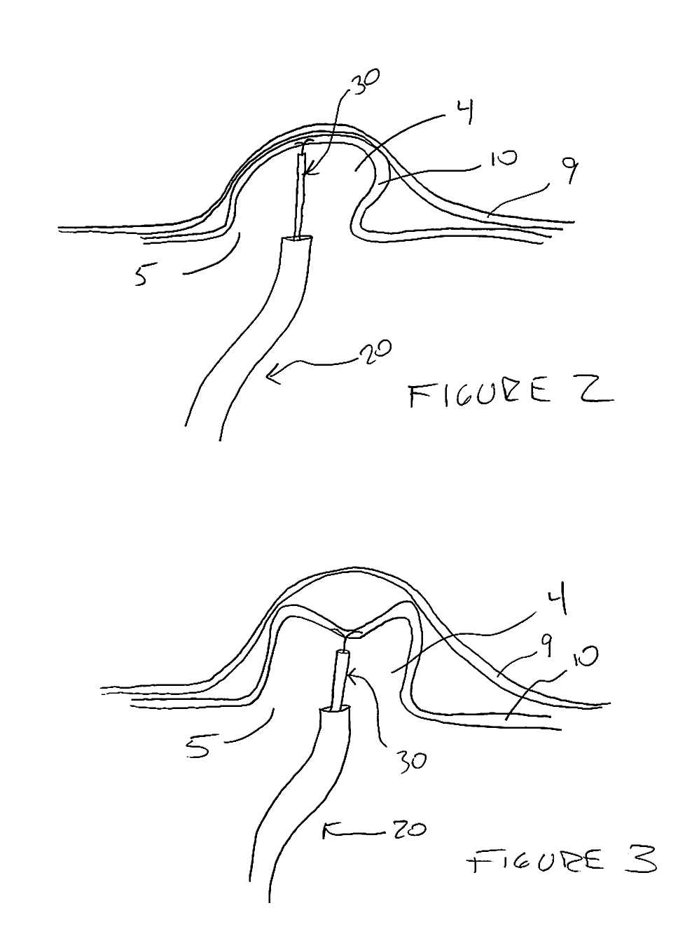

The invention includes many embodiments of methods and devices useable to create a separation between the wall 10 of the LAA 4 and the pericardium 9. Creating this separation is generally shown in FIGS. 2 and 3. As shown in FIG. 2, the catheter 20 has been navigated to the ostium 5 of the LAA 4. A tissue engaging device 30 has been deployed into the LAA 4 and has engaged the tissue of the distal wall 10 of the LAA 4. In FIG. 3, the tissue engaging device 30 is being retracted into the catheter 20, thereby creating a separation between the pericardium 9 and the LAA wall 10.

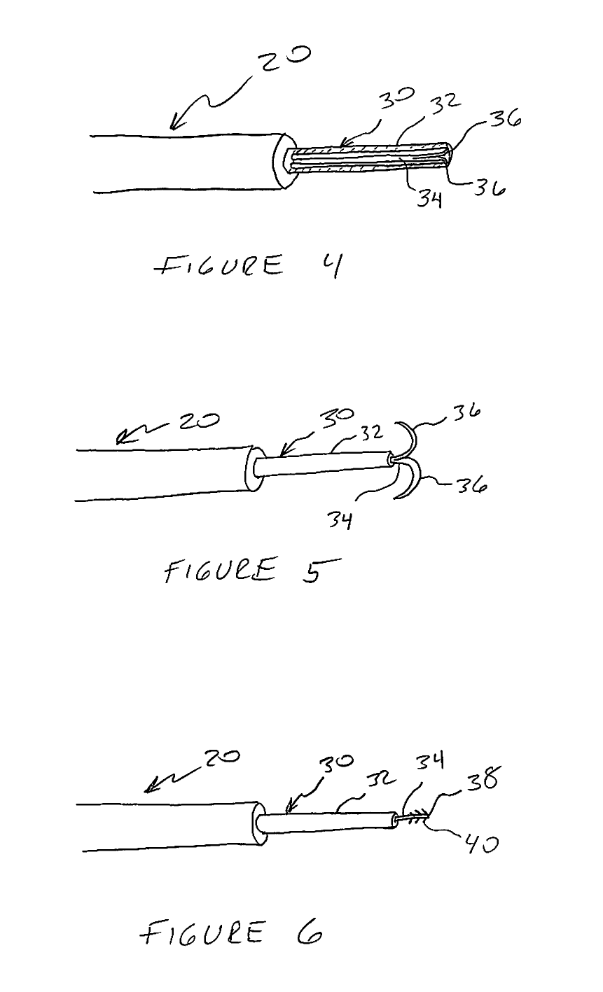

FIGS. 4 and 5 show an embodiment of a tissue engaging device 30 in a retracted state (FIG. 4) and a deployed state (FIG. 5). The tissue engaging device 30 is slidably disposed within the catheter 20 and includes a sheath 32 and an engagement tool 34. FIG. 4 shows the sheath 32 axially cutaway so the engagement tool 34 is visible in its contained configuration. The engagement tool 34 is a wire-like device having an end that includes at least one hook 36. The at least one hook 36 is predisposed to a deployed, curved configuration, as shown in FIG. 5, but is flexible enough to assume a straight configuration when contained inside the sheath 32. The radius of the curve of the hook 36 is selected such that when the engagement tool 34 is extended relative to the sheath 32, the axial advancement of the hook 36 is enough to engage tissue of the LAA wall 10 but flares outwardly prior to reaching the pericardium 9. The number of hooks 36 is at least one but may be two, as shown, or even three or more. The sliding relationship between the sheath 32 and the engagement tool 34 may also be limited to prevent the tool 34 from being able to reach the pericardium 9.

Alternatively, as shown in FIG. 6, the engagement tool 34 may be constructed with a barbed distal end 38 including at least one barb 40. In this embodiment, the engagement tool 34 is slidingly disposed within the sheath 32 such that the distance it may be extended is limited to prevent the engagement tool 34 from being able to puncture and/or engage the pericardium 9.

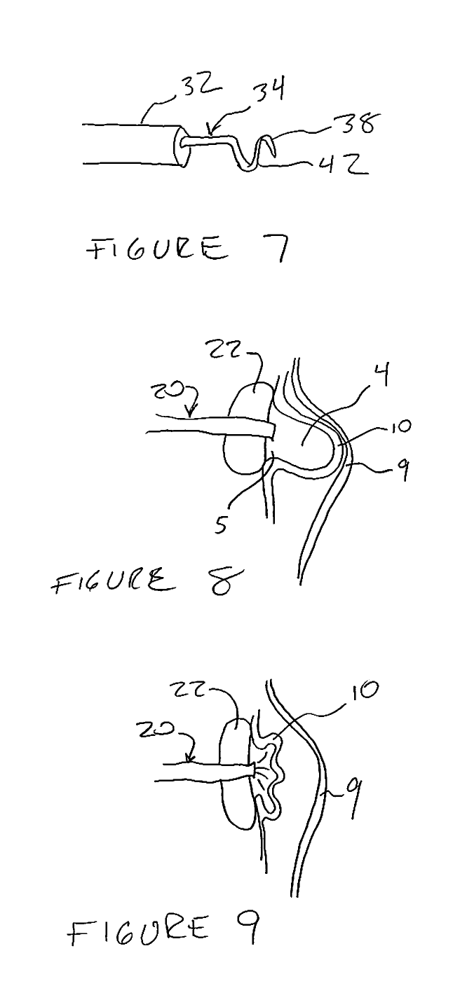

FIG. 7 shows another embodiment of the distal end 38 of the engagement tool 34 in which a helical screw tip 42 is usable to engage the wall 10 of the LAA 4. The engagement tool 34 of this embodiment is both slidingly contained within the sheath 32 but also able to be rotated relative to either the sheath 32 or the catheter 20.

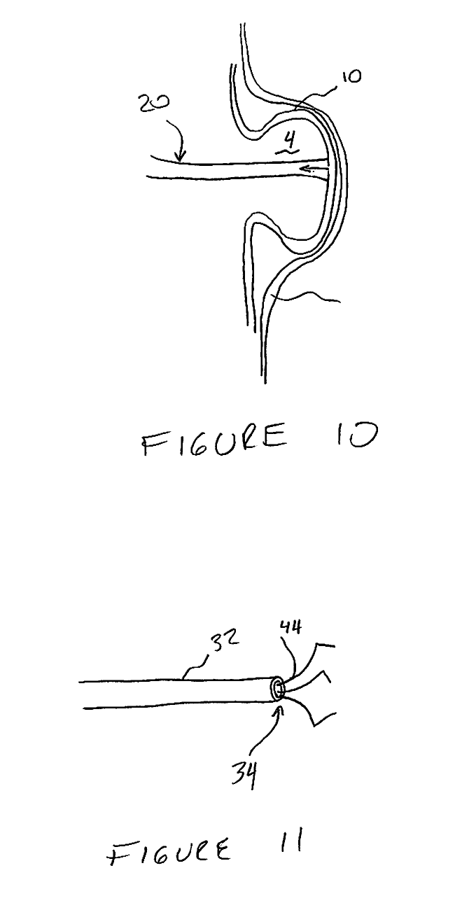

FIGS. 8 and 9 show yet another embodiment useable to retract the wall 10 in order to create space between the wall 10 and the pericardium 9. In this embodiment the catheter 20 includes a distal balloon 22 sized and shaped to be able to create a seal over the ostium 5 of the LAA 4. As shown in FIG. 9, once inflated, suction may be applied to remove the blood from inside the LAA 4, creating a vacuum inside the LAA 4 strong enough to collapse the wall 10 and maintain the wall 10 in a collapsed state while penetrating the wall 10 with a distal retraction device, discussed below.

It is also envisioned to use the catheter 20 as the engagement tool. This embodiment involves advancing the catheter 20 into the LAA 4 until the distal open end of the catheter 20 contacts the wall 10. Suction is then applied to the wall 10 using a suction lumen in order to retract the wall 10 away from the pericardium 9. It may be advantageous to employ a slightly flared distal catheter end in order to provide a better seal against the wall 10. To this end, may also be advantageous to use a softer material at the distal tip of the catheter 20.

FIG. 11 shows another embodiment of the distal end 38 of the engagement tool 34 including a plurality of graspers 44 that curve outwardly when advanced from a sheath 32. The graspers are capable of penetrating the tissue of the wall 10 but are not able to extend all the way through the wall 10. When the sheath 32 is advanced back over the graspers 44, the graspers close toward each other, thereby gripping the tissue and allowing the wall 10 to be retracted.

Deploying the Distal Retraction Device

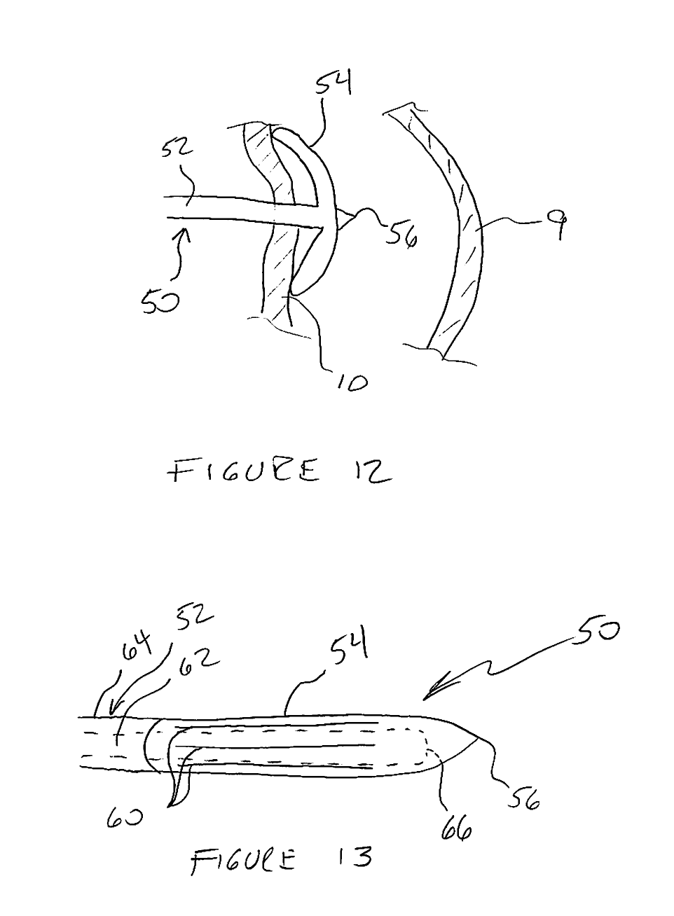

The next step in the method of the present invention is to puncture the distal wall 10 of the LAA 4 and deploy a distal retraction device 50 between the LAA wall 10 and the pericardium 9. This step is generally shown in FIG. 12. The distal retraction device 50 generally includes a shaft 52 and an expandable component 54, various embodiments of which are described below. The distal wall 10 of the LAA 4 is preferably held away from the pericardium 9 with the engagement tool 34 (not shown in FIG. 10), or the vacuum in the case of the embodiment of FIGS. 8 and 9, while the wall is being punctured by the device 50 to maintain the space between the wall 10 and the pericardium 9, and to provide proximal resistance to the distal force exerted on the wall 10 while it is being punctured by the device 50. A sharp distal tip 56 may be provided to effect puncturing the wall 10.

FIG. 13 provides an example of an embodiment of a distal retraction device 50 of the invention. In this embodiment, distal retraction device 50 includes a shaft 52 that includes a pusher sheath 64 slidably disposed over an inner element 62. The inner element extends into the expandable component 54 and has a distal terminus 66 that is attached to the distal tip 56 of the expandable component 54. The expandable component 54 includes an inner lumen containing the inner element 62 and a plurality of longitudinal slits 60.

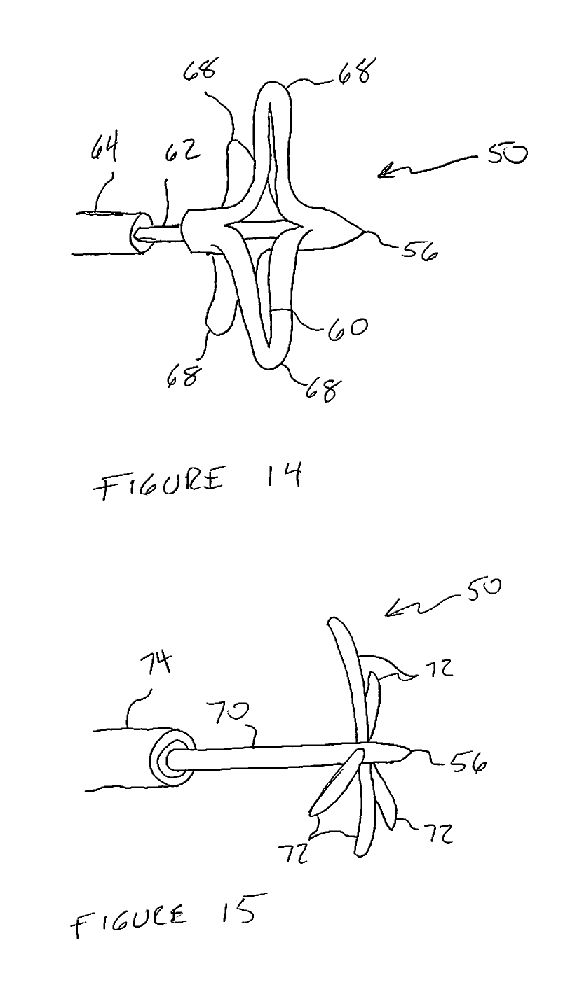

As seen in FIG. 14, when the pusher sheath 64 is advanced distally, relative to the inner element 62, the length of the expandable component 54 is shortened, causing the expandable component 54 to buckle, utilizing the slits 60 as relief. The result is a plurality of bent arms 68 radiating from the shortened expandable component 54. The pusher sheath 64 is shown retracted slightly to show the inner element 62.

FIG. 15 shows another embodiment of a distal retraction device 50. This device 50 includes a shaft 70 to which a plurality of self-radiating arms 72 extend, not unlike an umbrella. A deployment sheath 74 contains the shaft 70 and the arms 72 in a collapsed configuration until the distal tip 56 punctures through distal wall 10 of the LAA 4 and the device 50 can be deployed. At this time the sheath 74 is retracted relative to the shaft 70, allowing the arms 72 to radiate outwardly. In one embodiment, the arms are stored in the sheath 74 such that the ends 76 of the arms 72 are at the distal end of the device 50. This embodiment allows deployment almost immediately after the wall 10 has been penetrated. In another embodiment, the arms 72 are stored in the sheath 74 such that the ends 76 extend proximally in the sheath 74. This embodiment requires more space behind the wall 10 before the arms are deployed, such that the arms do not get fouled by the tissue of the wall 10. However, this embodiment may result in a greater retractive force being placed on the arms without the arms reassuming a folded configuration.

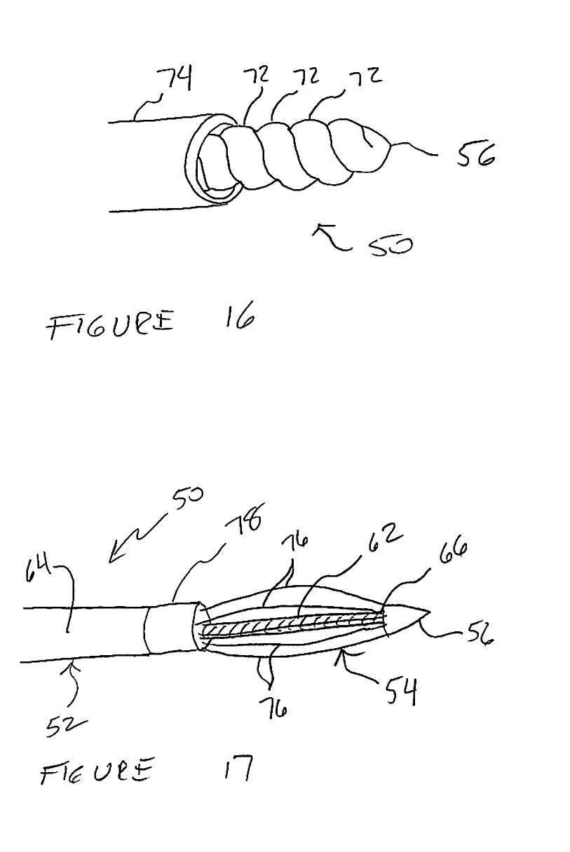

It is also envisioned that the arms 72 may be contained in the sheath 74 in a spiral configuration, as shown in FIG. 16. This embodiment provides the additional strength of rearward-collapsing arms while minimizing the axial advancement past the distal wall 10 needed for deployment.

Another embodiment of a distal retraction device 50 is shown in FIG. 17. Like the embodiment of FIG. 13, this distal retraction device 50 includes a shaft 52 that includes a sheath 64 slidably disposed over an inner element 62. The inner element extends into the expandable component 54 and has a distal terminus 66 that is attached to the distal tip 56 of the expandable component. The expandable component 54, however, includes a plurality of wires 76 that extend between a proximal gathering cuff 78 and the distal tip 56. These wires 76 may be a self-expanding material, such as Nitinol, or they may be actively expanded by shortening the distance between the distal tip 56 and the proximal gathering cuff 78. Shortening this distance may be accomplished by applying a relative distal force on the sheath 64. Alternatively, the proximal gathering cuff 78 may be constructed to act against a distal surface of the wall 10 when the distal retraction device 50 is retracted.

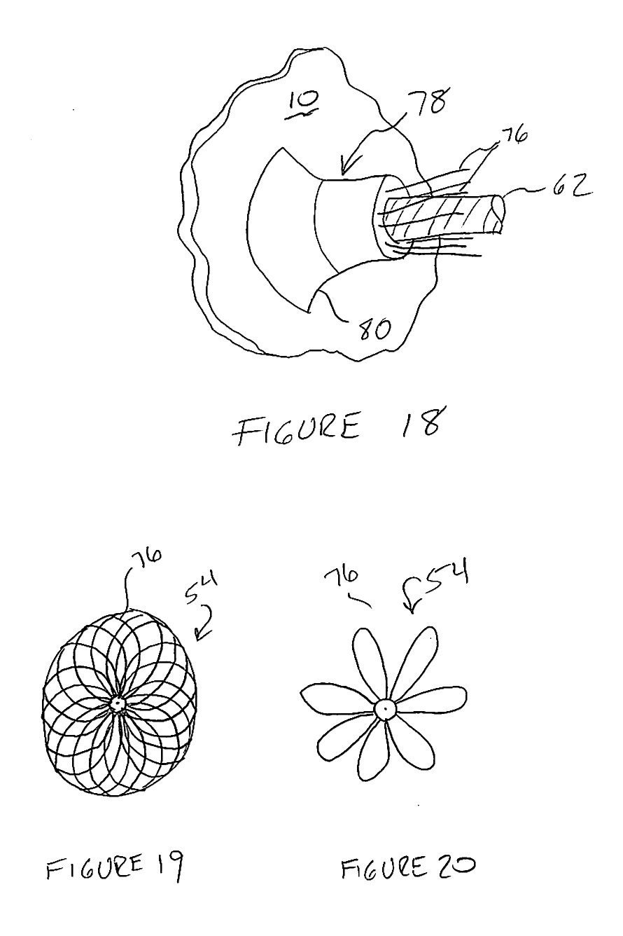

For example, FIG. 18 depicts a proximal gathering cuff 78 that includes a flexible skirt 80. The flexible skirt 80 flares outwardly when acting against the wall 10 of the LAA. The skirt 80 thus provides the proximal resistance necessary to expand the expandable component 54. The skirt 80 also serves to contain any potential bleeding through the small penetration made in the wall 10.

It is also envisioned that the proximal gathering cuff, with or without the skirt 80, be treated with a compound, such as a medicament, fibrin, fibrinogen, polymer, or a hydrogel, for example, that further prevents bleeding and promotes healing of the penetration through the wall 10. Alternatively or additionally, a membrane may be used to cover any of the expandable components of the invention in order to create a better seal.

Additionally, the wires 76 of the embodiment shown in FIG. 17 may be straight, braided, looped, or any configuration that would result in an expanded configuration when deployed. For example, FIG. 19 shows an axial view of an expandable component 54, in the expanded configuration that results from braided wires 76. FIG. 20 shows an axial view of an expandable component 54 in the expanded configuration that results from straight wires 76.

Reducing the LAA Volume

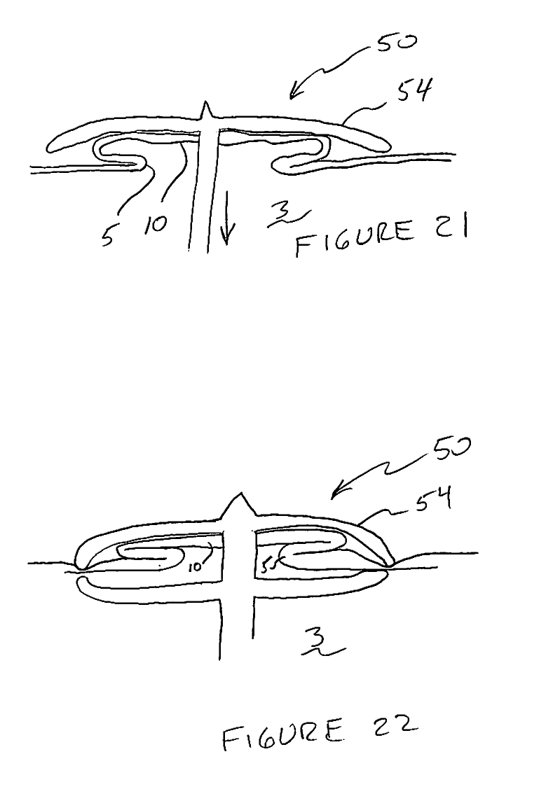

The next step in the method of the present invention is to reduce or eliminate the volume of the LAA by retracting the wall 10 toward the ostium 5 and securing the wall 10 such that the wall 10 blocks the ostium 5. As seen in FIG. 21, this is accomplished by pulling the distal retraction device 50, thereby moving the wall 10 toward the atrium 3 until the ostium 5 is blocked. Preferably, the expandable component 54 of the distal retraction device 50 is sized such that it is greater than the diameter of the ostium 5, thereby providing the physician a tactile indication that the expandable component 54 has contacted the outer surface of the left atrium and preventing the LAA from becoming inverted into the left atrium.

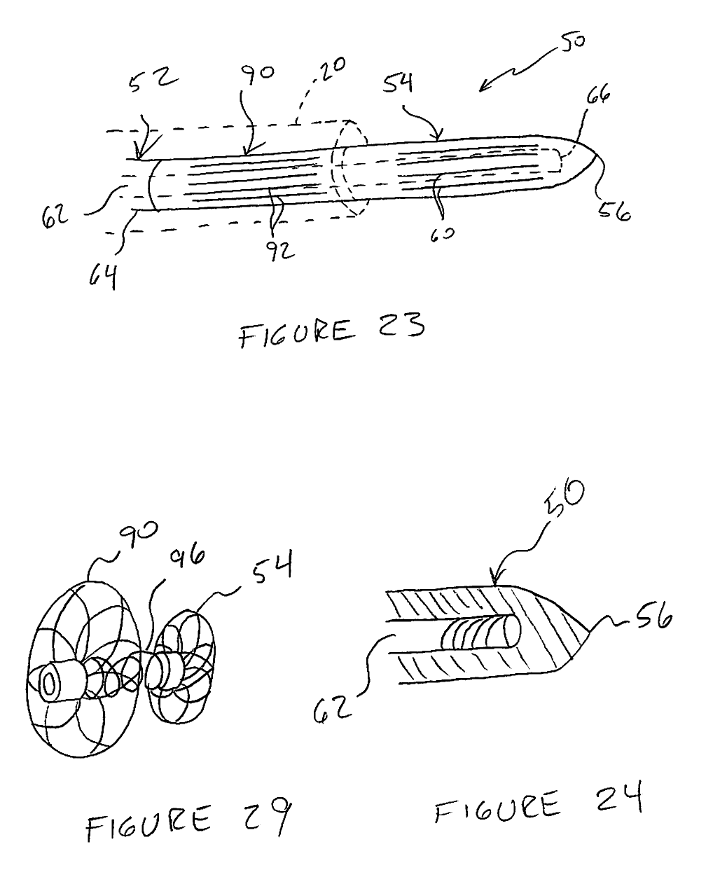

Once the wall 10 has been retracted to the ostium 5, it must be secured in place. One embodiment of the invention, shown in FIG. 22, deploys a proximal anchor 90 to accomplish this. The proximal anchor 90 may have a design similar to any of the designs of the expandable component 54 of the distal retraction device 50. The proximal anchor 90 may even be an integral component of the distal retraction device 50. For example, as shown in FIG. 23, there is shown a distal retraction device 50, similar to that shown in FIG. 13, including a shaft 52 that includes a pusher sheath 64 slidably disposed over an inner element 62. The inner element extends into the expandable component 54 and has a distal terminus 66 that is attached to the distal tip 56 of the expandable component 54. The expandable component 54 includes an inner lumen containing the inner element 62 and a plurality of longitudinal slits 60. A second set of slits 92 form the proximal anchor 90.

In this embodiment, the expandable component 54 and the proximal anchor 90 are both expanded by pushing the device 50 while pulling on the inner element 62. In order to selectively expand the expandable component 54 first, a sheath is placed over the device. Preferably, in order to save space, the catheter 20 may be used as a containment sheath. As shown in phantom lines in FIG. 23, the catheter 20 is positioned to allow the expandable component 54 to buckle and expand, while preventing the proximal anchor 90 from expanding. Thus, once the distal wall 10 has been retracted to the ostium 5, the proximal anchor may be deployed by retracting the catheter 20 in order expose the proximal anchor 90. Once exposed, the proximal anchor may be expanded in the same way the expandable component 54 was expanded.

It is to be understood that any of the various embodiments of the expandable component 54 may also be used as the proximal anchor 90. Additionally, any combination of components could be used. For example, the braided wire embodiment of the expandable component 54 shown in FIG. 19 may be combined with the slit tube embodiment of the proximal anchor 90 shown in FIG. 23; the proximal anchor 90 may have a straight wire construction like the one shown in FIG. 20 while the expandable component 54 may have a braided wire design or vice versa, etc. It is also to be understood that the dimensions of the expandable component 54 and the proximal anchor 90 do not have to be related to each other.

An embodiment utilizing braided wires on both the expandable component 54 and the proximal anchor 90 is shown in FIGS. 28 and 29. This embodiment also utilizes a connecting spring 96 between the expandable component 54 and the proximal anchor 90. The connecting spring 96 may be included in any of the embodiments shown and described herein, and may be helpful in lessening stress on the tissue during contraction of the heart. In other words, the spring 96 acts as a shock absorber as the tissue flexes and the blood pressure in the atrium 3 fluctuates.

Releasing the Device

Once the proximal anchor 90 has been expanded, it is necessary to release the device 50 so that the catheter 20 may be removed. As shown in FIG. 24, one embodiment includes a threaded connection between the inner element 62 and the distal tip 56. The threaded tip 56 would allow the inner element to be unscrewed from the device 50 once the device is in place. Risk of premature release is minimized because until the device is expanded, the device would rotate with the inner element 62. Once the expandable component 54 and the proximal anchor 90 are expanded, the contact between these components and the tissue provided enough counter-rotating force that the threaded tip 56 can be unscrewed from the device 50.

In either of the surgical embodiments, releasing the device can be accomplished by cutting the connector mechanically under direct access, or by any of the described methods.

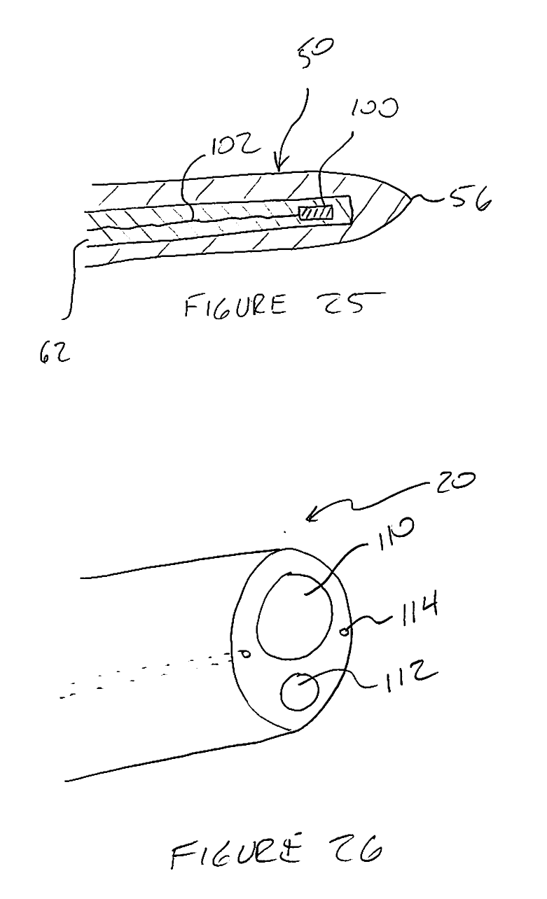

Another embodiment, shown in FIG. 25, provides a small heating element 100 in the distal end of the inner element 62 and a lead 102 supplying power to the heating element 100. The inner element 62 may be joined to the device 50 with an adhesive that breaks down when heated. When the physician is satisfied with the deployment of the device 50, power is supplied to the heating element 100, thereby degrading the adhesive and releasing the inner element 62 from the device 50.

Catheter Design

The catheter 20 preferably houses and delivers all of the components discussed herein. One embodiment of the delivery catheter 20 is shown in FIG. 26. The catheter 20 includes a primary lumen 110, a secondary lumen 112 and at least one steering wire 114. The primary lumen 110 is sized to accommodate the device 50 while the secondary lumen is sized to accommodate the tissue engaging device 30. Alternatively, if suction is used to engage the tissue, as discussed above, the secondary lumen may be utilized as a suction lumen.

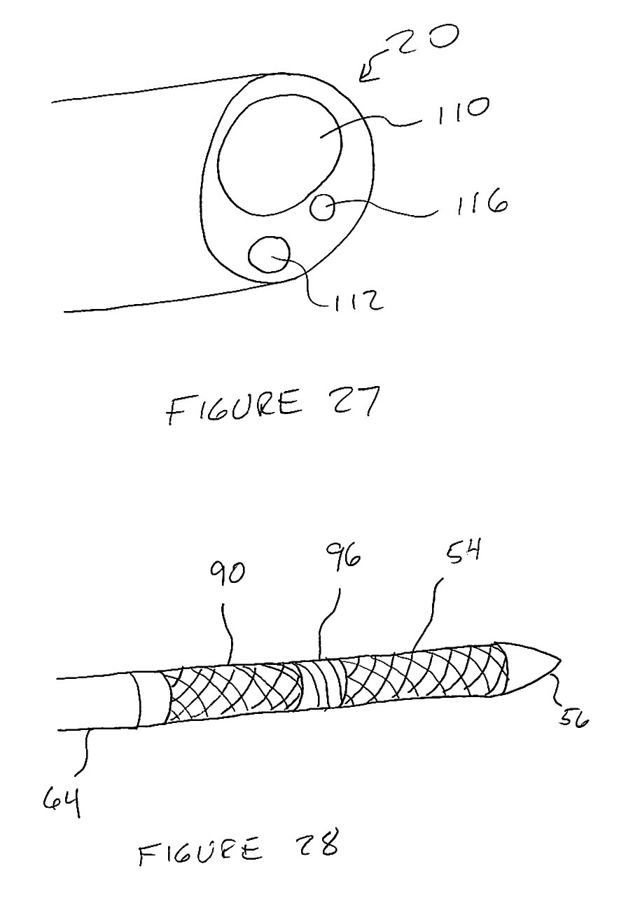

FIG. 27 shows another embodiment of a non-steerable delivery catheter 20 of the present invention. This embodiment includes a primary lumen 110 and a secondary lumen 112, as described above, and a guidewire lumen 116 usable to advance the catheter 20 over a guidewire. It is also envisioned that the tissue engagement device 30 may be incorporated into the end of a guidewire, in which case the secondary lumen 112 may be used as a guidewire lumen.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

For example, the devices and methods disclosed herein may be supplemented with features such as physiologic parameter monitoring. An example of this would be the incorporation of a pressure sensor mounted on the left atrial side of the device 50. The pressure sensor is constructed and arranged to continuously or sporadically measure left atrial pressure. Additionally or alternatively, an electrocardiographic or electrical sensor could be placed on the left atrial side of the device 50 so that acute or chronic electrophysiology parameters can be obtained. Other parameters that may be monitored include, but are not limited to, left atrial size and function, left ventricular size and function, flow into and through the mitral valve, the occurrence of atrial fibrillation, the sensing of arrhythmias such as supraventricular arrhythmias or ventricular arrhythmias, to name just a few. These sensing device may be attached to or may become integral to the device 50.

What is claimed is:

1. A method of treating a left atrial appendage of a patient comprising:
accessing the left atrial appendage via the left atrium of a patient;
deploying an expandable device between the left atrial appendage and the pericardium of the patient adjacent the left atrial appendage;
reducing an internal volume of the left atrial appendage by retracting the tissue of the left atrial appendage toward the ostium of the left atrial appendage;
securing the expandable device such that said internal volume of the left atrial appendage remains reduced;
releasing said expandable device.

2. The method of claim 1 wherein accessing the left atrial appendage comprises navigating a catheter to the left atrial appendage via the left atrium of a patient.

3. The method of claim 2 wherein deploying an expandable device between the left atrial appendage and the pericardium of the patient adjacent the left atrial appendage comprises creating a space between the left atrial appendage and the pericardium of the patient adjacent the left atrial appendage.

4. The method of claim 3 wherein navigating a catheter to the left atrial appendage via the left atrium of a patient comprises navigating said catheter through the foramen ovale of the patient.

5. The method of claim 3 wherein creating a space between the left atrial appendage and the pericardium of the patient adjacent the left atrial appendage comprises grabbing tissue of a distal wall of the left atrial appendage and retracting said tissue proximally.

6. The method of claim 3 wherein creating a space between the left atrial appendage and the pericardium of the patient adjacent the left atrial appendage comprises applying a suction force to tissue of a distal wall of the left atrial appendage and retracting said tissue proximally.

7. The method of claim 3 wherein creating a space between the left atrial appendage and the pericardium of the patient adjacent the left atrial appendage comprises creating a seal over the ostium of the left atrial appendage and creating suction inside the left atrial appendage, resulting a collapse thereof.

8. The method of claim 3 wherein deploying an expandable device inside said space comprises releasing a self-expanding device inside said space.

9. The method of claim 3 wherein deploying an expandable device inside said space comprises releasing a mechanically-expandable device inside said space.

10. The method of claim 3 wherein deploying an expandable device inside said space comprises expanding said device by shortening a length thereof.

11. The method of claim 3 wherein reducing an internal volume of the left atrial appendage by retracting the tissue of the left atrial appendage toward the ostium of the left atrial appendage comprises pulling said expandable device toward the ostium.

12. The method of claim 3 wherein reducing an internal volume of the left atrial appendage by retracting the tissue of the left atrial appendage toward the ostium of the left atrial appendage comprises blocking the ostium of the left atrial appendage with said tissue.

13. The method of claim 3 wherein securing the expandable device such that said internal volume of the left atrial appendage remains reduced comprises expanding a proximal anchor in the left atrium that spans the ostium.

14. The method of claim 1 wherein reducing an internal volume of the left atrial appendage by retracting the tissue of the left atrial appendage toward the ostium of the left atrial appendage comprises using the tissue of the left atrial appendage as a barrier against blood entering the left atrial appendage.

15. A device for blocking the ostium of the left atrial appendage of a patient with tissue of the left atrial appendage comprising an expandable component connected to a proximal anchor, said expandable component deployable between a wall of the left atrial appendage and the pericardium adjacent to the wall and usable to pull the wall against the ostium, the proximal anchor having a diameter greater than that of the ostium and usable to maintain the wall in place against the ostium.

16. The device of claim 15 wherein said expandable component comprises a tube with a plurality of slits cut therein such that upon shortening said tube, said slits cause sections of said tube therebetween to buckle outwardly.

17. The device of claim 15 wherein said expandable component comprises a plurality of wires arranged to bend radially outwardly when said expandable component is shortened.

18. The device of claim 15 wherein said expandable component self-expands when released from a sheath.

19. The device of claim 15 wherein said proximal anchor comprises a tube with a plurality of slits cut therein such that upon shortening said tube, said slits cause sections of said tube therebetween to buckle outwardly.

20. The device of claim 15 wherein said proximal anchor comprises a plurality of wires arranged to bend radially outwardly when said expandable component is shortened.

21. The device of claim 15 wherein said proximal anchor self-expands when released from a sheath.

22. The device of claim 15 further comprising a spring connecting said expandable component and said proximal anchor.

23. A system for reducing a volume of the left atrial appendage of a patient comprising:
a delivery catheter having at least one lumen;
a tissue engaging device associated with said delivery catheter; and,
a releasable device slidably disposed within said lumen;
wherein said tissue engaging device is usable to create space between a distal wall of the left atrial appendage and adjacent pericardial tissue and said releasable component includes an expandable component deployable in said space and usable to secure tissue of said wall against the ostium of the left atrial appendage, thereby preventing blood from flowing through the ostium into the left atrial appendage.

24. The system of claim 23 wherein said delivery catheter comprises a primary lumen and a secondary lumen, said primary lumen containing said releasable device and said secondary lumen containing said tissue engaging device.

25. The system of claim 23 wherein said delivery catheter comprises a primary lumen and a suction lumen, said primary lumen containing said releasable device.

26. The system of claim 23 wherein said tissue engaging device comprises a sheath and an engagement tool slidably disposed within said sheath.

27. The system of claim 26 wherein said engagement tool has an end that includes a feature selected from the group consisting of: hook, barb, screw, suction tube and grasper.

28. The system of claim 23 wherein said releasable device comprises an expandable component connected to a proximal anchor, said expandable component deployable between a wall of the left atrial appendage and the pericardium adjacent to the wall and usable to pull the wall against the ostium, the proximal anchor having a diameter greater than that of the ostium and usable to maintain the wall in place against the ostium.

29. The system of claim 28 wherein said expandable component comprises a tube with a plurality of slits cut therein such that upon shortening said tube, said slits cause sections of said tube therebetween to buckle outwardly.

30. The system of claim 28 wherein said expandable component comprises a plurality of wires arranged to bend radially outwardly when said expandable component is shortened.

31. The system of claim 28 wherein said expandable component comprises an expandable component self-expands when released from a sheath.

32. The system of claim 28 wherein said proximal anchor comprises a tube with a plurality of slits cut therein such that upon shortening said tube, said slits cause sections of said tube therebetween to buckle outwardly.

33. The system of claim 28 wherein said proximal anchor comprises a plurality of wires arranged to bend radially outwardly when said expandable component is shortened.

34. The system of claim 28 wherein said proximal anchor self-expands when released from a sheath.

35. The system of claim 28 further comprising a spring connecting said expandable component and said proximal anchor.
